(19) United States
(12) Patent Application Publication
(10) Pub. No.: US 2016/0184564 A1
(43) Pub. Date: Jun. 30, 2016

(54) INTRANASAL DELIVERY DEVICE AND METHOD OF MATERIAL DELIVERY

(71) Applicants: Michael R. Spearman, The Woodlands, TX (US); John Henry Burban, Lake Elmo, MN (US); John William Shanahan, White Bear Lake, MN (US); Keith A. Roberts, Dellwood, MN (US)

(72) Inventors: Michael R. Spearman, The Woodlands, TX (US); John Henry Burban, Lake Elmo, MN (US); John William Shanahan, White Bear Lake, MN (US); Keith A. Roberts, Dellwood, MN (US)

(21) Appl. No.: 14/586,962

(22) Filed: Dec. 31, 2014

(51) Int. Cl. A61M 31/00

(52) U.S. Cl. CPC A61M 31/00

(57) ABSTRACT

A delivery device for a method of targeted delivery of materials to nasal tissue uses:
a carrier layer having a first surface and an opposed second surface for providing the material from the first surface of the carrier layer onto the nasal tissue in contact with the first surface of the carrier layer;
a support layer contiguous with at least a portion of the opposed second surface;
wherein the combination of the carrier layer and the support layer enabling pressure to be exerted or increased between the first surface of the carrier layer and the nasal tissue in contact with the first surface of the carrier layer.

### BACKGROUND OF THE INVENTION

1. Field of the Invention

The present invention relates to the field of material delivery, devices for material delivery, material delivery to nasal tissue and methods for material delivery to nasal tissue.

2. Background of the Art

There is an entire facet of the medical industry that is dedicated to methods and devices for delivery of materials to patients. Delivery is made both for inactive (dyes, pigments, contrast enhancers, etc.) as well as for medically active ingredients. Delivery has been performed by oral administration, topical application (solutions, dispersions, suspensions, cremes) sprays, injections, infusion, intravenous delivery and the like. The method of delivery tends to be formulated on the basis of the nature of a condition that is being addressed, the location of the condition, the amount and type of medical material needed to be used in the treatment, and the relative effectiveness of the type, location and amount of treatment.

For many conditions, nasal delivery of ingredients is appropriate. The most typical method of nasal delivery is topical, especially by spray and drops. Another method of nasal delivery is through the use of nasal splint, nasal tampons, nasal packing or nasal sponges. These terms tend to be relatively equivalent within the art. Among the various conditions which can be at least partially addressed by nasal administration include, but are not limited to sinusitis, rhinitis, headaches (including migraines and cluster headaches), nasal congestion, allergic reactions, anxiety, asthma, and numerous other conditions. By addressing tissue structure and mucosal release, beneficial impact on snoring may also be effected.

For example, an analgesic compound (e.g. a non-opioid analgesic peptide, an NOP agonist or N/OFQ (Nociceptin/orphanin FQ (N/OFQ), a 17-amino acid neuropeptide, is the endogenous ligandfor the nociceptin receptor (NOP, ORL-1). It is derived from the prepronociceptin protein, as are a further 2-peptides, nocistatin and NocII) or a pharmaceutical composition comprising an analgesic compound (e.g. a non-opioid analgesic peptide, an NOP agonist or N/OFQ) may be dispensed intranasally as a powdered or liquid nasal spray, suspension, nose drops, a gel, film or ointment, through a tube or catheter, by syringe, by packtail, by pledget (a small flat absorbent pad), by nasal tampon or by submucosal infusion. Nasal drug delivery can be carried out using devices including, but not limited to, unit dose containers, pump sprays, droppers, squeeze bottles, airless and preservative-free sprays, nebulizers (devices used to change liquid medication to an aerosol particulate form), metered dose inhalers, and pressurized metered dose inhalers. It is important that the delivery device protect the drug from contamination and chemical degradation. The device should also avoid leaching or absorption as well as provide an appropriate environment for storage. Each drug needs to be evaluated to determine which nasal drug delivery system is most appropriate. Nasal drug delivery systems are known in the art and several are commercially available.

Typical devices and methods include material delivered to the inferior two-thirds of the nasal cavity by direct placement of the composition in the nasal cavity, for example, with a gel, an ointment, a nasal tampon, a dropper, or a bioadhesive strip. Alternatively, an analgesic compound (e.g. a non-opioid analgesic peptide, an NOP agonist or N/OFQ) or a pharmaceutical composition comprising an analgesic compound (e.g. a non-opioid analgesic peptide, an NOP agonist or N/OFQ) can be delivered to the superior region of the nose, or both the superior region and inferior region.

Typical commercial NSAIDS (non-steroid anti-inflammatant drugs) include:

Aspirin (Anacin®, Ascriptin®, Bayer®, Bufferin®, Ecotrin®, Excedrin®)

Choline and magnesium salicylates (CMT, Tricosal®, Trilisate®)

Choline salicylate (Arthropan®)

Celecoxib (Celebrex®)

Diclofenac potassium (Cataflam®)

Diclofenac sodium (Voltaren®, Voltaren XR®)

Diclofenac sodium with misoprostol (Arthrotec®)

Diflunisal (Dolobid®)

Etodolac (Lodine®, Lodine XL®)

Fenoprofen calcium (Nalfon®)

Flurbiprofen (Ansaid®)

Ibuprofen (Advil, Motrin, Motrin IB, Nuprin)

Indomethacin (Indocin®, Indocin SR®)

Ketoprofen (Actron®, Orudis®, Orudis KT®, Oruvail®)

Magnesium salicylate (Arthritab®, Bayer Select®, Doan's Pills®, Magan®, Mobidin®, Mobogesic®)

Meclofenamate sodium (Meclomen®)

Mefenamic acid (Ponstel®)

Meloxicam (Mobic®)

Nabumetone (Relafen®)

Naproxen (Naprosyn®, Naprelan*®)

Naproxen sodium (Aleve®, Anaprox®)

Oxaprozin (Daypro®)

Piroxicam (Feldene®)

Rofecoxib (Vioxx®)

Salsalate (Amigesic®, Anaflex 750®, Disalcid®, Marthritic, Mono-Gesic, Salflex, Salsitab®)

Sodium salicylate (various generics)

Sulindac (Clinoril®)

Tolmetin sodium (Tolectin®)

Valdecoxib (Bextra®)

Other materials and methods known for nasal delivery are indicated below.

U.S. Pat. No. 8,481,043 (Bergenhem) discloses compositions and methods for intranasal delivery of antigens for immunization of a mammal. Antigens include peptides, proteins, peptidomimetics, DNA, RNA, carbohydrates and phospholipids. The compositions contain at least one antigen and a permeation enhancer. The permeation enhancer can be a macrocyclic permeation enhancer, such as a Hsieh enhancer. The pharmaceutical composition may be administered intranasally in the form of a gel, an ointment, a nasal emulsion, a lotion, a cream, a nasal tampon, or a bioadhesive strip. The nasal delivery device can be metered to administer an accurate effective dosage amount to the nasal cavity. The nasal delivery device can be for single unit delivery or multiple unit delivery. The compounds of the Bergenhem invention may also be delivered through a tube, a catheter, a syringe, a packtail, a pledget, a nasal tampon or by submucosal infusion (e.g., U.S. Patent Publication Nos. 20090326275, 20090291894, 20090281522 and 20090317377).

U.S. Pat. No. 7,074,426 (Kochinke) relates to methods of treating various orofacial diseases involving inflammation, infection and/or pain, using intratissue controlled release drug delivery systems. More particularly, the invention relates to methods for localized or targeted administration of a sustained release formulation of an agent such as an anti-inflammatory agent to a specified tissue location within the orofacial environment. Other drug delivery target structures can be, for example and not limited to, the brain (1-dopa in Parkinson's disease); inflamed nasal mucous membranes that are lining the passages of the nose due to, for example, seasonal allergic, perennial allergic, or perennial nonallergic rhinitis (anti-histamines and/or steroids); eye muscles in exophthalmos, which is the condition in which one or both of the eyes bulge out of their sockets as a consequence of Grave disease, and which can cause loss of eye muscle control, double vision, and may require specialized treatment with steroids; and so forth.

U.S. Pat. Nos. 7,655,244 and 7,749,515 (Blumenfeld) disclose that botulinum toxin, among other presynaptic neurotoxins is used for the treatment and prevention of migraine and other headaches associated with vascular disorders. Presynaptic neurotoxins are delivered focally, targeting the nerve endings of the trigeminal nerve, the occipital nerve and the intranasal terminals of the parasympathetic fibers originating in the Sphenopalatine ganglion. The administration preferably targets the extracranial nerve endings of the trigeminal nerve in the temporal area, the extracranial occipital nerve endings in the occipital area, and the intranasal terminals of the trigeminal nerve and parasympathetic fibers originating in the Sphenopalatine ganglion. The delivery is carried out by way of injection or topically. Nociceptive fibers stimulated by inflammatory mediators in infectious or allergic rhinitis can also activate the trigeminal nerve.

U.S. Pat. Nos. 7,691,394, 7,670,608 and 8,679,486 (Borodic) provide improved formulations of botulinum toxin that increase delivery of the botulinum toxin to neural and associated tissues and exhibit a higher specific neurotoxicity and higher potency (in LD₅₀ Units) than available formulations of botulinum toxins. These improved formulations enable physicians to treat a wide variety of pathological conditions with a lower toxin load that reduces the risk of inducing an immune response against the toxin and its associated proteins that may ultimately lead to the development of toxin resistance. These benefits are particularly important in the treatment of conditions that require high-dose or chronic administration of botulinum toxin. Additionally, the decrease in LD₅₀ Unit doses of inventive formulations allows for controlled administration which limits diffusion. The Borodic inventions also provide methods of treating neuromuscular diseases and pain, using low-dose botulinum toxin. Rhinitis treatment is also identified.

U.S. Pat. No. 7,749,515 (Blumenfeld) also provides improved formulations of botulinum toxin that increase delivery of the botulinum toxin to neural and associated tissues and exhibit a higher specific neurotoxicity and higher potency (in LD₅₀ Units) than available formulations of botulinum toxins. These improved formulations enable physicians to treat a wide variety of pathological conditions with a lower toxin load that reduces the risk of inducing an immune response against the toxin and its associated proteins that may ultimately lead to the development of toxin resistance. These benefits are particularly important in the treatment of conditions that require high-dose or chronic administration of botulinum toxin. Additionally, the decreased in LD₅₀ Unit doses of inventive formulations allows for controlled administration which limits diffusion. The present invention also provides methods of treating neuromuscular diseases and pain, using low-dose botulinum toxin. Some devices have been developed which position a portion of the middle turbinate against the nasal septum prior to endoscopic surgery. This type of nasal splint increases visualization of the nasal cavities to facilitate nasal endoscopic surgery and protects a portion of the middle turbinate from endoscopic tools during surgery by moving or repositioning the middle turbinate against the nasal septum. Unfortunately, these devices require being secured to the nasal septum as well as moving the middle turbinate against the nasal septum which may not be feasible if the turbinate is large or swollen which could lead to blockage of nasal breathing. For examples, see U.S. Pat. No. 5,599,284, “Pre-operative nasal splint for endoscopic sinus surgery and method” and U.S. Pat. No. 5,713,839 (Shea), “Pre-operative nasal splint for endoscopic sinus surgery and method”. Another drawback to these types of splints is that it may not be possible to reposition the turbinate without fracturing it.

Turbinate splints have also been developed and are commonly used as nasal post-operative devices, for example, see U.S. Pat. No. 5,350,396 (Eliachar). These types of splints address particular problems encountered after nasal septal reconstructive surgery. They are usually utilized to support the septum in the correct position during healing and recovery after surgery. Other types of post-operative turbinate splints have been an asset in insuring a more complete separation of the nasal mucosal membranes after surgery or injury, but none of the post-operative nasal splints are configured to be utilized during FESS and FTSI procedures to protect a nasal turbinate from trauma or damage from surgical instruments.

U.S. Pat. Nos. 7,879,340 (Sanders) discloses a method and composition for blocking or reducing physiological reaction in a mammal to the interaction of IgE antibodies present in said mammal upon contact with the corresponding antigen, by the administration to said mammal of a therapeutically effective amount of a neurotoxin (CnT) derived from Clostridia sp. A method is disclosed of blocking or reducing allergic rhinitis in a mammal resulting from the interaction of IgE antibodies present in said mammal upon contact with the corresponding antigen, by the administration to said mammal of a therapeutically effective amount of a neurotoxin (CnT) to treat allergic rhinitis, wherein the CnT is isolated or purified from a species of Clostridia selected from the group consisting of C. botulinum, C. butyricum and C. beratti. Topical treatment of allergic rhinitis includes steroid sprays and chromalyn sodium (Nasocrom®) a chemical that blocks mast cell degranulation, and/or nasal decongestants (Neosynephrine). Systemic treatment includes oral anti-histamines and non-sedating antihistamines (Allegra®, Zyrtec®, Claritin®). Long-term therapy requires immune desensitization to the allergen by progressive intradermal injections of the allergen over months to years.

U.S. Pat. No. 6,436,950 (Achari) describes intranasal delivery methods and compositions for the delivery of dopamine receptor agonists which are effective for the amelioration of erectile dysfunction in a mammal without causing substantial intolerable adverse side effects to the mammal. Nasally administered compositions for treating male erectile dysfunction in a mammal are also provided which include a therapeutically effective amount of a dopamine receptor agonist which has been dispersed in a system to improve its solubility and/or stability. The compositions according to the present invention can be administered, for example, as a nasal spray, nasal drop, suspension, gel, ointment, cream or powder. The administration of a composition can also include using a nasal tampon or a nasal sponge containing a composition of the Achari invention. The dopamine receptor agonist can also be brought into a viscous basis via systems conventionally used, for example, natural gums, methylcellulose and derivatives, acrylic polymers (carbopol) and vinyl polymers (polyvinylpyrrolidone). In the present compositions, many other excipients known in the art can be added such as preservatives, surfactants, co-solvents, adhesives, antioxidants, buffers, viscosity enhancing agents and agents to adjust the pH and the osmolarity.

U.S. Pat. No. 8,551,949 (Toll) provides for methods and compositions for treatment of pain via craniofacial mucosal administration of an analgesic compound (e.g. a non-opioid analgesic peptide, an NOP agonist or N/OFQ). Intranasal administration of certain analgesic peptides such as N/OFQ results in global analgesic effects. In addition, the nasal delivery system of the pharmaceutical composition can include a buffer to maintain the pH of the dopamine receptor agonist, a pharmaceutically acceptable thickening agent and a humectant. The pharmaceutical composition can further include one or more pharmaceutical excipients and even further include a pharmaceutically acceptable preservative. The buffer of the nasal delivery system can be selected from the group including acetate, citrate, prolamine, carbonate and phosphate buffers. The thickening agent of the nasal delivery system can be selected from the group including methyl cellulose, xanthan gum, carboxymethyl cellulose, hydroxypropyl cellulose, carbomer, polyvinyl alcohol, alginates, acacia, chitosans and combinations thereof. The humectant of the nasal delivery system can be selected from the group including sorbitol, glycerol, mineral oil, vegetable oil and combinations thereof. The methods including treating erectile dysfunction in a male mammal including nasally administering a pharmaceutical composition including a therapeutically effective amount of a dopamine receptor agonist in combination with a nasal delivery system wherein the pharmaceutical composition does not cause substantial intolerable adverse side effects in the mammal.

U.S. Pat. Nos. 7,879,340, 8,088,360 and 8,349,292 (Sanders) provide examples of addenda in solutions for nasal delivery such as oxidized cellulose (marketed as Surgicel® by Johnson & Johnson, New Brunswick, N.J.). The oxidized cellulose can be manufactured with BoNT (e.g., Botox Type-A)as an integral component, or the BoNT can be added to the oxidized cellulose before its clinical use. Surgicel® polymer is available in the form of a thin flexible sheet that is often cut to fit the area of the body to which it will be applied. For intranasal use the size may vary from a few square millimeters to a 4 by 8 cm² sheet that could contact the entire exposed mucosa of the nasal cavity. BoNT can be added to the Surgicel® polymer as a lyophilized powder or after reconstitution into solution. If added as a powder it can homogeneously applied onto one side of the Surgicel® polymer and the material can be folded. The material is then moistened with normal saline to bind the material together and immobilize the BoNT prior to clinical use. In contrast the BoNT can first be constituted into solution and then absorbed into the material.

U.S. Pat. No. 8,241,641 (Blumenfeld) discloses that Botulinum toxin, among other presynaptic neurotoxins is used for the treatment and prevention of migraine and other headaches associated with vascular disorders. Presynaptic neurotoxins are delivered focally, targeting the nerve endings of the trigeminal nerve, the occipital nerve and the intranasal terminals of the parasympathetic fibers originating in the Sphenopalatine ganglion. The administration preferably targets the extracranial nerve endings of the trigeminal nerve in the temporal area, the extracranial occipital nerve endings in the occipital area, and the intranasal terminals of the trigeminal nerve and parasympathetic fibers originating in the Sphenopalatine ganglion. The delivery is carried out by way of injection or topically. In a preferred embodiment, the presynaptic neurotoxin of the invention is Botulinum toxin. In a particularly preferred embodiment of the invention, the presynaptic neurotoxin is Botulinum toxin A. Botulinum toxin A is presently supplied and commercially available as “Botox”® by Allergan, Inc. of Irvine, Calif., and as “Dysport”® by Ipsen, of Berkshire, UK. In another embodiment of the invention, the presynaptic neurotoxin is Botulinum toxin B. Botulinum toxin B is commercialized as “Neurobloc”®/“Myobloc”(R) by Solstice Neuroscience, Inc, of San Francisco, Calif. A pentavalent toxoid of all eight known Botulinum serotypes is also available as an investigational drug from the U.S. Center for Disease Control in Atlanta, Ga. The Botulinum A toxin preparations are most preferred for their known safety and efficacy. Botox® has also been used to treat, among other things, cervical dystonia, brow furrows, blepharospasm, strabismus, and hyperhydrosis. To facilitate administration, the presynaptic neurotoxins will most preferably be formulated in unit dosage form. The presynaptic neurotoxins may be supplied, for example, as a sterile solution or as a lyophilized powder in a vial. In a preferred embodiment of this invention, the presynaptic neurotoxins are administered by way of injection. However, in other embodiments, the presynaptic neurotoxins may be administered topically. In general, the preparation of the presynaptic neurotoxin solution for topical delivery may be the same as that which is injected. However, in other embodiments, the presynaptic neurotoxin may be applied topically via a carrier known to those of skill in the art. The solutions can then be administered by several means, like for example, a pledget of cotton or cotton tipped applicator, a dropper or a spray in the case of a solution, or a spatula in the case of a cream. These topical methods of application may be used wherever trigeminal, occipital or parasympathetic nerve endings can be accessed efficiently by such application. The solution containing the presynaptic neurotoxins can be administered topically to the epidermis through these means and the presynaptic neurotoxins can then be distributed through the epidermis by transdermal carrier systems. As would be obvious to one of skill in the art, topical administration may not be as effective as administration via injection depending on, for example, the patient, the severity of the symptoms and access to the trigeminal, occipital and parasympathetic nerves. To target the intranasal trigeminal nerve endings and parasympathetic nerve endings in the nasal mucosa, the method of administration involves infiltrating the upper respiratory tract (nasal mucosa and turbinates) with a presynaptic neurotoxin diluted with a suitable solution such as saline. There is a coalescence of nerve fibers within the upper region of the nose (intranasally) near the cribiform plate and above the superior turbinate. It is also known anatomically that the inferior turbinate, which is the most responsive organ in the nose, is formed of bone and mucosa composed of vascular lakes that provide the basis for nasal sprays and topical medications administered intranasally which provide for rapid local transmucosal absorption.

In a preferred embodiment, Botulinum toxin Type A (Botox® toxin) is used, and each nostril is infiltrated with 5 to 10 units using a solution of 100 units of Botox® toxin diluted with 4 cc of normal saline. The infiltration in this preferred embodiment is performed by injection using a 30 gauge needle. Intranasal injections are given in each nostril using endoscopic application, or a needle palpation technique. The needle is inserted through the nostril. Lateral and medial mucosal infiltration is performed either through finger palpation and guidance or through direct visualization using a nasal speculum and an external light source or via endoscopic guidance.

In another embodiment, Botox® toxin is used and it is administered in the region of the external nares using 5 to 10 units using a solution of 100 units of Botox® diluted with 4 cc of normal saline. The infiltration in this preferred embodiment is performed by injection using a 30 gauge needle. If necessary, Botox® can be also administered in the distribution of the infraorbital nerve.

In another embodiment of the invention, infiltration of the presynaptic neurotoxins in the upper respiratory tract will be done by topical administration to the intranasal mucosa (either alone or with a carrier substrate) which, because of its anatomical proximity to the end terminals of the trigeminal nerve or the sphenopalatine ganglion that innervate the nose, will have a direct effect on the alleviation of headache pain. In a particular embodiment, the presynaptic neurotoxin solution, with or without a carrier, will be delivered with a cotton pledget or a dropper and spread along the targeted area. In another embodiment of the invention, the presynaptic neurotoxin solution, with or without a carrier, will be delivered in the form of an emollient, cream or solution, and spread over the epidermis of the targeted area. In yet a different embodiment of the invention, the presynaptic neurotoxin solution, with or without a carrier, will be delivered in the form of a spray. Other embodiments may use alternative methods of topical delivery known to those of skill in the art.

All documents cited herein are incorporated by reference in their entirety.

### SUMMARY OF THE INVENTION

A delivery device is enabled for targeted delivery of materials to nasal tissue. The device may have:
a carrier layer having a first surface and an opposed second surface for providing the material from the first surface of the carrier layer onto the nasal tissue in contact with the first surface of the carrier layer;
a support layer contiguous with at least a portion of the opposed second surface of the carrier layer; and
the combination of the carrier layer and the support layer enabling pressure to be exerted or increased between the first surface of the carrier layer and the nasal tissue in contact with the first surface of the carrier layer.

The delivery device may have delivery capability for a full 360° of its circumference or may be designed for targeted for limited delivery over (for example) 240°, 200°, 180°, 120° or less of targeted delivery from the circumference of the carrier layer of the device.

### BRIEF DESCRIPTION OF THE FIGURES

FIG. 1 shows a side view of a basic delivery device of the present invention with an indicated degree of expansion upon absorbance of liquid.

FIG. 2 shows a side view of a delivery device having a fluid delivery conduit before expansion.

FIG. 3 shows a side view of a delivery device having a fluid delivery conduit after expansion.

FIG. 4 shows a side view of a delivery device having a fluid delivery conduit before expansion of a partially enclosing carrier layer over a structural support element.

FIGS. 5A and 5B show a perspective, exploded view of other insert device structures with up to 3 layers and up to 5 layers, respectively.

FIG. 6A shows a perspective view of a carrier-support combination before and after addition of water.

FIG. 6B shows a perspective view of a controlled dimension carrier-support combination before and after addition of water.

### DETAILED DESCRIPTION OF THE INVENTION

A delivery device is enabled for targeted delivery of materials to nasal tissue. The device may have:
a carrier layer having a first surface and an opposed second surface for providing the material from the first surface of the carrier layer onto the nasal tissue in contact with the first surface of the carrier layer;
a support layer contiguous with at least a portion of the opposed second surface of the carrier layer; and
the combination of the carrier layer and the support layer enabling pressure to be exerted or increased between the first surface of the carrier layer and the nasal tissue in contact with the first surface of the carrier layer. In identifying that the “ . . . combination of the carrier layer and the support layer enabling pressure to be exerted or increased between the first surface of the carrier layer and the nasal tissue in contact with the first surface of the carrier layer . . . ”, the present technology allows for the enabling pressure to be provided by the carrier layer alone, the support layer alone, or both the support layer and carrier layer in combination. This also includes use of an expandable layer between the support layer and the carrier layer.

As described in greater detail, imbibing fluids into the respective layers can cause expansion and creation of expansion pressure, filling internal balloon elements with fluids can cause expansion of the device, mechanical lever-like elements or springs or elastic memory in components can cause expansion pressure, and these may be positioned within the article in various positions and on or in various layers to effect this outward pressure application with the device.

The materials carried by the carrier layer will be described in more detail later, but they may include inactive materials (e.g., dyes and pigment for image enhancement, isotopes to enhance imaging, and the like), medically inert but active materials (e.g., saline solution, sugar solutions, oils, emollients, fragrances, odor antagonists, and other materials that are not intended for a specific medical effect, etc.) and medically active ingredients. These medically active ingredients will be the most thoroughly analyzed of the materials useful in the practice of the present technology.

The delivery device at some point during its use will most likely contain a liquid material the addition of which liquid material to the carrier and/or support layer has expanded the volume of the carrier and/or support layer. The expansion will expand the dimensions of the carrier layer towards the nasal tissue. The liquid may expand the carrier and/or support layer by either swelling compositional material of the carrier and/or support layer (e.g., fibers, gels, foams, fabrics, and the like) or the liquid may by thickening action or viscosity of the added liquid (such as a gel, foam, highly viscous but flowable liquid, etc.) add internal pressure to the carrier layer and/or support to expand the carrier and/or support layer. The liquid may be added before, during or after insertion of the device into the desired position in the nasal area where the material is to be delivered. Even if pressure is to be applied by means other than introduction of liquids to the carrier and/or support layer, the carrier and/or support layer may contain a dry powder that is pressed into contact with controlled pressure against the nasal tissue.

The delivery device may further have an expanding element that provides force against an interior surface of the carrier layer to expand volume of the carrier layer or to provide the contact force between the carrier layer and the nasal tissue, such as a balloon, hydraulic lifting platform and the like. The expanding element may further be selected from a group consisting of inflatable elements, elements with an expansive elastic memory (springs, compressed accordion elements, sponges, compressible foams (with or without surface absorption capability), and clip-like elements where applied force to one segment of the clip-like element expands another segment of the element, such as with levered clips, bag-closure clips and the like which are commercially available. The expansion may be manually or mechanically controlled and maintained during application of materials from the device, and the expansion (and inherent pressure against tissue surfaces) may be stopped to facilitate timely removal of the device.

The shape, construction, imbued actives content and available processes can assist in not only the unmet need in the market for targeting delivery, but also controlling an appropriate dose in a correct location, and minimizing amounts of dose material in the application and use of the device so as to control costs. As the costs of conventional doses prescribed and used for some of these agents tends to be very high and use of large amounts are often needed to assure that a needed minimum amount of the total dose is appropriately delivered in a generally desired area, the controlled delivery (in both location and amounts) facilitates conservation of supplies, use of lower amounts of actives in the actual delivery system, and minimizing overall costs, as compared to many conventional delivery systems for these types of actives.

The delivery device may be constructed, especially by selection of compositions and amounts of components in the carrier and/or support layer and amounts and components in the liquid added, such that the addition of the liquid material has expanded or expands the volume of the carrier and/or support layer by at least 5%, at least 10% or even at least 20% to exert or increase pressure between the first surface of the carrier layer and the nasal tissue. Actual expansion will be used in certain environments in degrees over 50%, over 75% and up to 100% or more as needed.

An alternative method in the delivery device for increasing or creating pressure between the carrier layer and nasal tissue would be by providing a conduit for delivery of expanding fluid pressure such that it is provided contiguous (directly against or through an intermediate layer) to the second surface of the carrier layer. There are alternative structures, later described in greater detail in the Figures, wherein the delivery device has on the second surface of the carrier layer a fluid pressure barrier layer adhered thereto (or may be a coating or fusion of material at the inner surface of the carrier layer or a distinct layer positioned or adhered against that inner surface) and the conduit provides fluid pressure on a side of the fluid pressure barrier layer distal from the second surface of the carrier layer to cause exertion of or increase of pressure between the first surface of the carrier layer and the nasal tissue in contact with the first surface of the carrier layer. The relative sizes of the layers will depend upon specific effects desired in the use of the device of the present invention. The support layer may have the same, smaller or larger dimensions than the carrier layer. The relative thicknesses of the layers may vary, with the carrier layer initially the same, larger or smaller thickness than the support layer.

The expansion of the carrier layer by direct introduction of fluid into the carrier layer or expansion may be over a support element having structural features dividing areas thereon. The expansion of the carrier layer by fluid pressure caused by delivery of a fluid on the distal side of the barrier layer may be present between the distal surface of the barrier layer and the support layer or the support layer has a first surface and an opposed second surface and at least a portion of the opposed second surface that is not in contact with any portion of the carrier layer (that is, only one side of the support is actually carrying deliverable material. For example, the support layer has a first surface and an opposed second surface and at least 25% of the opposed second surface is not in contact with any portion of the carrier layer. In such fluid pressure delivery systems, the support layer has a distinct first surface and an opposed distinct second surface having a distinct structural feature (e.g., a channel, edge, rib, panel, etc.) separating the distinct first surface and the opposed distinct second surface and at least 25% of the opposed second surface is not in contact with any portion of the carrier layer. The delivery device may have the structural feature divide the support layer into the distinct first surface and the distinct second opposed surface and the distinct first surface comprises between 25% and 80% of total surface area formed by combined total surface areas of the distinct first surface and the opposed distinct second surface. This creates a defined area of carrier layer than can be raised away from the support layer or support element in a limited direction (e.g., in a single side or limited portion of the nasal area within which the device has been positioned.

The delivery device preferably has the structural feature separate a zone contiguous with the first distinct surface of the support layer within which the fluid pressure expanding fluid pressure is provided to expand the carrier layer or cause the carrier layer to exert pressure against the nasal tissue.

A method of delivery material to be absorbed into or through nasal tissue comprising adding material to a delivery device comprising:
a carrier layer having a first surface and an opposed second surface for providing the material from the first surface of the carrier layer onto the nasal tissue in contact with the first surface of the carrier layer;
a support layer contiguous with at least a portion of the opposed second surface of the carrier layer;
the combination of the carrier layer and the support layer enabling pressure to be exerted or increased between the first surface of the carrier layer and the nasal tissue in contact with the first surface of the carrier layer;
inserting the delivery device into a nasal passage and exerting pressure or increasing pressure between the first surface of the carrier layer and the nasal tissue while material to be delivered is present within the carrier layer.

The method may have the pressure exerted or increased by addition of fluid (gas, liquid or gel) into the carrier and/or support layer, or by expansion of a mechanical element such as an expanding living hinge, expanding clip, screw-levered hinge and the like. Therefore, the material of the carrier and/or support layer need not always be of a composition that expands by physical absorption of fluids, but alternatively may be mechanically or hydraulically expandable or may serve to transfer the pressure force from a mechanical element. The fluid added to the carrier layer may contain the material carried in the fluid to be delivered to the nasal tissue. Alternatively, the fluid added to the carrier layer assists delivery of material already within the carrier layer before addition of the liquid onto or into the nasal tissue. In the preferred method, the material comprises medically active ingredients.

In the step of inserting the delivery device into a nasal passage and exerting pressure or increasing pressure between the first surface of the carrier layer and the nasal tissue while material to be delivered is present within the carrier layer, this pressure may, as previously noted, be mechanical, hydraulic, or expansive (e.g., due to swelling), and the originating force may come from any one or combinations of layers and additional elements.

Without being limited to the specific classes or species of drugs listed below, these are examples of the types of medically active ingredients that are specifically included within the scope and objectives of the present technology. The medically active ingredients may, for example be selected from the group consisting of analgesics, antibiotics, antifungal agents, antihistamines, NSAIDS, steroids, botulinum toxin, Clostridia sp derived neurotoxin, vaccine, viruses, bacteria, liposomes, noisomes and tetanus toxins.

The present system uses local administration of the ingredients for local, or systemic effective activity. As used herein ‘local administration’ includes but is not limited to topical administration in lyophilized powder, liquid solutions, creams, ointments, or introduced by liposomal (niosomes) vectors, or as nucleic acid introduced by viral or other vectors. The medical material may also be embedded in biopolymers which release the medical materials into solution over prolonged or predetermined periods. The device of the present technology is most generally used by implantation of the device and physical removal of the device, partial removal (of the support layer) and subsequent removal of the carrier layer or partial removal (of the support layer) and subsequent dissolution of the carrier layer.

By “therapeutically effective amount” it is meant of purposes of this invention that the medically active ingredient is administered in a non-toxic amount sufficient to cause reduction in the occurrence or magnitude of the symptoms being targeted.

By “unit” it is meant the biological equivalent of the current unit measure used for medically active ingredients, especially such as botulinum toxin A marketed as Botox. At present BoNT is measured by biological assay; a unit of BoNT is the amount that causes death to 50% of mice when injected intraperitoneally. BoNT-A is marketed as Botox by Allergan Corp, Irvine Calif., and as Dysport™ by Ipsen Ltd, Berks United Kingdom. Although the biological assay is done the same way the in vivo effect of Botox and Dysport™ vary. BoNT-B is marketed as Myobloc™ by Elan Pharmaceuticals, Dublin, Ireland. TeNT (tetanus neurotoxin) is not commercially available but other assays have compared the potency of the blocking effect of TeNT to BoNT. All serotypes of BoNT as well as TeNT are commercially available from List Biological laboratories. A therapeutically effective amount of BoNT will vary depending on the organ to be treated, how much of the organ will be treated, the method of application and the exact preparation of BoNT used. A therapeutically effective amount will vary from a fraction of a unit to hundreds of units as it currently does with intramuscular injections. The exact dosage will not require undo experimentation by those skilled in the art.

Where solutions or suspensions of medically active ingredient are referred to, unless indicated to the contrary, this means the designated number of units in 1 ml of Normal saline or water, especially deionized water.

By “CnT” it is meant that any biological substance having essentially the same biological effect within cells as the wild types of clostridia neurotoxins, specifically, to block or decrease the activity of the SNARE family of proteins (Soluble N-ethylmaleimide-sensitive factor activating protein receptor) involved in secretion of allergy related neurohumors. Numerous substitutions for the major parts of the CnT have been disclosed and these are all included in this specification. This would include fragments, altered forms, and recombinant forms of CnT. Also included are chimeras, hybrids and conjugates. Also included are the use of DNA and RNA sequences that are directly applied and translated in the allergic sites. Also included are “vectors”, various compositions that deliver a botulinum or tetanus toxin light chain or its equivalent such as Protease A across cell membranes. These vectors include but are not limited to viruses, liposomes, noisomes, and protein transduction domains.

Allergic “neurohumors” are neurotransmitters, neuropeptides and cytokines that participate in allergic reactions and whose secretion or action can be blocked by CnT. They include acetylcholine, noradrenaline, neuropeptide Y, substance P, calcitonin gene reactive protein (CGRP), histamine, nerve growth factor, and interleukins. This technology is directed to a method of blocking or reducing physiological reaction in a mammal, suitably but not limited to H. sapiens, to the interaction of IgE antibodies present in said mammal upon contact with the corresponding antigen. This blocking is achieved by the administration to said mammal of a therapeutically effective amount of a neurotoxin (CnT) derived from Clostridia sp. Suitably, the CnT is derived from a species of Clostridia selected from the group consisting of C. botulinum, C. butyricum, C. beratti, C. tetani. The neurotoxins (BoNT), derived from C. botulinum, are derived from serotypes A, B, C1, D, E, F and G, while neurotoxin (TeNT), is derived from C. tetani

BoNT/A is marketed as Botox® by Allergan Inc and as Dysport® by Ipsen Ltd as a lyophilized powder that is reconstituted with preservative free normal saline prior to use. BoNT/B is marketed as Myobloc® by Elan Pharmaceuticals in normal saline solution. The light chains and holotoxins for each BoNT serotypes and TeNT can be obtained from List Biological Labs and/or Metabiologics Inc.

Medically active ingredient compositions of the present inventions are prepared in a variety of forms depending on whether the composition is implanted, topically applied to respiratory mucosa of the nasal cavity and coated by the device or allowed to migrate through tissue in contact with the carrier layer.

For all applicable medically active ingredient compositions fluid dosage forms are prepared utilizing the compound and a pyrogen-free sterile vehicle. The compound, depending on the vehicle and concentration used, can be either dissolved, dispersed or suspended in the vehicle. In preparing solutions the compound can be dissolved in the vehicle, the solution being made isotonic if necessary by addition of sodium chloride and sterilized by filtration through a sterile filter using aseptic techniques before filling into suitable sterile vials or ampoules and sealing. Alternatively, if solution stability is adequate, the solution in its sealed containers may be sterilized by autoclaving. Advantageously additives such as buffering, solubilizing, stabilizing, preservative or bactericidal, suspending or emulsifying agents and/or local anaesthetic agents may be dissolved in the vehicle.

Dry powders, which are dissolved or suspended in a suitable vehicle prior to use, or loaded dry into the material of the carrier layer, may be prepared by filling pre-sterilized drug substance and other ingredients into a sterile container using aseptic technique in a sterile area. Alternatively the drug and other ingredients may be dissolved into suitable containers using aseptic technique in a sterile area. The product is then freeze-dried and the containers are sealed aseptically.

In the respiratory systems, medically active ingredients bind to mucosal epithelial cells and are actively transcytosed across the mucosa. Compositions suitable for administration to the respiratory tract include gels, dissolvable powders, solutions, dispersions, suspensions, or micro-fine powders for insufflation. In the latter case, particle size of less than 50 microns, especially less than 10 microns, is preferred.

Compositions suitable for topical mucosal application include normal saline solutions as described above, gels and creams. Gels and creams tends to provide greater viscosity to the carrier layer and can more strongly support expansion pressure

The nasal tissue presents a modest barrier to the application of medically active ingredients such as CnT. The medically active ingredient may be mechanically propelled across the dermal barrier with air or water pressure injectors against a layer behind the carrier layer (distal from the support layer or fluid barrier layer) or in association with the carrier layer itself. Other suitable forms of transdermal delivery include iontophoresis across the carrier layer. The medically active ingredients may be encapsulated into liposomes or niosomes to form suitable trans-dermal compositions.

The method of administration may take many forms, including topical, intra-dermal, sub-cutaneous, trans-cutaneous, and intra cavital forms by inhalation of the medically active ingredients in a suitable carrier. Examples of such administration include, but are not limited to contact with absorbent pledgets (a component of the device having medically active ingredients adsorbed thereon or absorbed therein), contact with biodegradable micropellets in the carrier layer having medically active ingredients embedded therein. Alternatively less invasive methods include by drops into or spray of the medically active ingredient into the carrier layer before or after implantation.

The intranasal carrier may include ancillary ingredients to assist in performance of the expansion of the carrier layer or other physical effects such as including a buffer. The buffer pH is selected to enhance absorption of the medical material and to produce early resultant effects from administering the dosage unit to a nasal mucosa of the mammal. Preferably, an effect is produced within about 60, 45, 40, 15 or even within 5 minutes. The intranasal carrier of the intranasal dosage unit is preferably an aqueous solution. Further, the aqueous solution can be selected from the group including aqueous gels, aqueous suspensions, aqueous liposomal dispersions, aqueous emulsions, aqueous microemulsions and combinations thereof. Alternatively, the intranasal carrier of the intranasal dosage unit is a non-aqueous solution, suspension or dispersion. The non-aqueous solution can be selected from a group including non-aqueous gels, non-aqueous suspensions, non-aqueous liposomal dispersions, non-aqueous emulsions and non-aqueous microemulsions and combinations thereof. The intranasal carrier of the intranasal dosage unit can also be a combination of an aqueous solution and a non-aqueous solution.

Alternatively, the carrier of the intranasal dosage unit is a powder formulation. The powder formulation can be selected from a group including simple powder mixtures, powder microspheres, coated powder microspheres, liposomal dispersions and combinations thereof. Preferably, the powder formulation is powder microspheres. The powder microspheres are preferably formed from various polysaccharides and celluloses selected from the group including starch, methylcellulose, xanthan gum, carboxymethylcellulose, hydroxypropyl cellulose, carbomer, alginate polyvinyl alcohol, acacia, chitosans and combinations thereof

The intranasal dosage unit can also include an excipient having bio-adhesive properties. Preferably, the buffer of the intranasal dosage unit is selected to have a pH of from about 3 to about 10 and more preferably from about 3.5 to 7.0.

Preferably, the intranasal dosage unit includes a humectant. A humectant can be selected from the group consisting of soothing agents, membrane conditioners, sweeteners and combinations thereof.

The carrier and support compositions and materials may be selected from known available materials. The carrier and support material may be a single composition material, or may be blends or mixtures of material that provide a combination of properties necessary for the expanding or increasing of pressure of the carrier layer between the support layer and nasal tissue. The carrier and support material should be compatible with the material being delivered (especially with respect to hydrophilicity or hydrophobicity of the fluid carrier). The carrier and support may be in fabric form, paper form, foam structure, porous mass, microporous mass or any other convenient structure. Hydrogels, superabsorbent polymers (fibers and particles), controlled foaming compositions and other compositions known to swell in contact with liquids (e.g., water, aqueous solutions, hydrophilic liquids, oils, gels, hydrophobic liquids, etc.) are also suitable. Non-swellable materials such as polymer fibers, fiber film, membranes, foams, reticulated foams and structures, fabrics and the like are also suitable. Inorganic such as glass fabric, carbon fiber fabric and the like may be used to internally support the carrier or support composition in the carrier or support layers, repsectively. Examples of commercially available materials such as the hydrogels and superabsorbent materials (and their additives and addenda) are described in the following materials.

Hydrogels

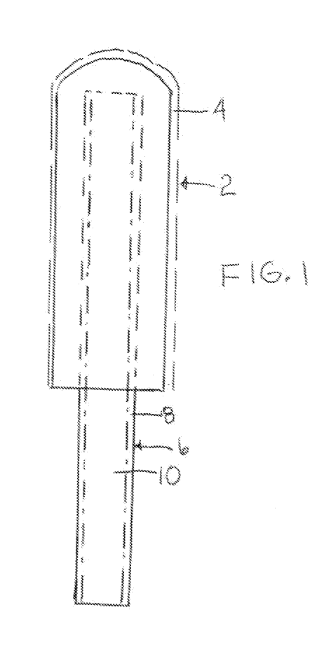

The hydrogel can be composed of collagen in one example (e.g., FIG. 1). The hydrogel can also be composed of keratins, fibrins, chitosans, glycosaminoglycans, elastins, matrigel, carrageenan, chondroitin sulfate, gelatin, pectin, alginate or gels based on combinations of these naturally occurring components), other naturally occurring or synthetic components comprising the gel can include fibronectins, laminins, proteoglycans, hyaluronan, glc-nac (N-Acetylglucosamine (N-acetyl-D-glucosamine, or GlcNAc, or NAG is a monosaccharidederivative of glucose. It is an amide between glucosamineand acetic acid. It has a molecular formula of C₈H₁₅NO₆), matricellular proteins (e.g., periostins and related polypeptides, CNN1, thrombospondins), collagen or elastin mimetics or combinations thereof, or synthetic hydrogel components (e.g. polyurethane hydrogel, PEG hydrogel, polyacrylate hydrogel, polyvinyl alcohol, sodium polyacrylate, acrylate polymers and copolymers, hydroxyethyl, carboxymethyl or carboxyethyl cellulose or combinations thereof), hybrid hydrogels synthetic or naturally occurring components (e.g., gels composed of collagen and polyethylene glycol (PEG)) and naturally occurring, synthetic or hybrid hydrogels functionalized with drugs, sugars, peptides, particles or other attached or integrated factors and the like may be used as the sole material or component material in the carrier layer. Non-swelling ingredients may be included to provide strength and/or control of the swellable component. For example, non-swelling polymeric or cellulose fibers and particles may be included in the carrier or support layer.

Other examples of hydrogel materials that can be used alone or in combination with other gel composite materials include polylactic glycolic acid-ethylene glycol-lactic glycolic acid, poly hydroxy butyrate, polypropylene fumarate-co-ethylene glycol, polylactic acid-ethylene glycol-lactic acid, polyethylene glycol-lactic acid-ethyleneglycol, polymethylmethacrylate-co-hyroxyethylmethacrylate, polyethylene glycol-bis-lactic acid-acrylate, polyethylene glycol-butylene oxide-terephtalate, polylactic glycolic acid-c-serine, polyhydroxypropylacrylamide-g-peptide, polyethylene glycol-g-acrylamide-co-vemine, polyethylene glycol/-cyclodextrins, polyvinylacetate/vinylalcohol, polyhydroxyethylmethacrylate/Matrigel, polyN-vinyl pyrrolidone, polyacrylonitrile-co-allyl sulfonate, polyethylene glycol dimethacrylate-sulfate, polyethyleneglycol-co-peptides), polybiscarboxy-phenoxy-phospazene, alginate-g-polyethylene oxide-propylene oxide-ethylene oxide, polyacrylamide, polyN-isopropyl acrylamide-co-acetic acid, polyN-isopropyl acrylamide-co-ethylmethacrylate, chitosan-g-polyethylene oxide-propylene oxide-ethylene oxide, hyaluronic acid-g-N-isopropyl polyacrylamide, alginate-acrylate and collagen-acrylate.

In another example, a hydrogel that can be activated to become fluid by lowering its temperature, to about 25° C. or below (i.e., room temperature). For example, such gels would be used to generate the tissue-engineered composition and then the gel depolymerized by temperature lowering so that the cellular elements of the composition that had been prompted by the methods described herein, to self-organize into the desired geometry, can be isolated from the hydrogel and then fused or otherwise combined to form new compositions as described herein to generate complex three- and two dimensional structures. One example of such multi-dimensional compositions would be tissue engineered organ or organ-like structures used in the regenerative repair of, for example damaged, diseased or congenitally malformed organs.

An example of a thermo-gel is a polymer of a meth-acrylamide derivative and a hydrophilic monomer. A further example of a thermogel hydrogel polymer is a biodegradable polymer containing a polyethylene glycol (PEG) block linked to biodegradable polyester. Other examples are gels based on pluronic acid that become liquid at 4° C. but form hydrogels at 37° C. These thermogels can also be mixed with other naturally occurring on synthetic hydrogel materials or contain other compounds that improve their performance in generating and maintaining the tissue engineered composition.

U.S. Pat. No. 8,809,301 (Athanasiadis); U.S. Pat. No. 6,312,725 (Wallace); U.S. Pat. No. 8,784,773; (Renani); and U.S. Pat. No. 7,834,065 Nakajima disclose surgical quality hydrogels, some of which are biodegradable and bioabsorbable.

U.S. Pat. No. 8,771,734 (Tabata) provides a sustained-release preparation which comprises a drug and a bioabsorbable polymer hydrogel, wherein a concentration gradient of the drug is formed in the hydrogel. Also disclosed is a method of sustained release of a drug in vivo using the sustained-release preparation of the invention. The directionality of the drug release may be controlled by employing the sustained-release preparation of the invention. The sustained-release preparation of the invention is particularly useful as an anti-cancer agent.

U.S. Pat. No. 8,750,988 (Jolly) describes a cochlear implant electrode includes an implantable electrode carrier having an outer surface with electrode contacts for electrically stimulating nerve tissue of the inner ear of a patient. A drug lumen is within the electrode carrier and adapted to receive a therapeutic fluid. The drug lumen contains delivery openings to the outer surface of the electrode carrier and a hydrogel matrix disposed between the drug lumen and the one or more delivery openings and adapted to swell in volume when exposed to the therapeutic fluid. The hydrogel matrix is adapted to control diffusion of the therapeutic fluid from the drug lumen through the delivery openings to the outer surface of the electrode carrier.

U.S. Pat. No. 8,735,374 (Zerbe) discloses a direct compression formulation suitable for preparing buccal and/or sublingual and dosage forms incorporating a combination of a non-ionic polymeric solubility enhancer, a mucoadhesive polymer, a filler, a disintegrant, and a pharmaceutically active agent. Cannabinoid-cyclodextrin complexes exhibiting an improved property selected from improved stability, higher product yield and improved product uniformity may be obtained by complexing the cannabinoid with the cyclodextrin in a liquid medium containing an antioxidant. To enhance stability, product yield and/or product uniformity, complexing may be done while the liquid medium is in contact with an atmosphere having a very low oxygen content. The resulting complexes may be combined with decomplexing agents and/or dispersed in a matrix material comprised of a hydrogel-forming polymer to provide enhanced absorption of the cannabinoid through oral mucosa and reduced ingestion of the cannabinoid as compared with known commercially available cannabinoid-containing oral dosage forms.

U.S. Pat. No. 7,098,194 (Chenite) discloses Methoxy PEG-succinoyl-N-hydroxysuccinimide ester (mPEG-suc-NHS), and methoxy PEG-carboxymethyl-NHS (mPEG-cm-NHS) that have been reacted with chitosan under homogeneous conditions in mild aqueous solution to produce hydrogel formulations. Such modified chitosan based formulations may form gels, at room temperature, within a few minutes depending upon the formulation characteristics.

Superabsorbent Polymers

SuperAbsorbent Polymers (also called SAPs) are polymers that can absorb and retain extremely large amounts of a liquid relative to their own mass. Water-absorbing polymers, which are classified as hydrogels when cross-linked, absorb aqueous solutions through hydrogen bonding with water molecules. An SAP's ability to absorb water is a factor of the ionic concentration of the aqueous solution. In deionized and distilled water, a SAP may absorb 500 times its weight (from 30 to 60 times its own volume) and can become up to 99.9% liquid, but when put into a 0.9% saline solution, the absorbency drops to maybe 50 times its weight. The presence of valence cations in the solution impedes the polymer's ability to bond with the water molecule.

The total absorbency and swelling capacityare controlled by the type and degree of cross-linkers used to make the gel. Low-density cross-linked SAPs generally have a higher absorbent capacity and swell to a larger degree. These types of SAPs also have a softer and stickier gel formation. High cross-link density polymers exhibit lower absorbent capacity and swell, but the gel strength is firmer and can maintain particle shape even under modest pressure.

U.S. Pat. No. 8,829,107 (Furno) discloses a method for the production of a superabsorbent composition comprising the process steps: i. production of a hydrogel by radical polymerization of an aqueous monomer solution containing at least one monomer ii. drying the hydrogel to obtain a water-absorbing polymer structure iii. surface crosslinking of the water-absorbing polymer structure to obtain a surface-crosslinked water-absorbing polymer structure, and iv. incorporating a starch compound into the method. The Furno technology also relates to a superabsorbent composition obtainable according to this method, a particulate superabsorbent composition and a composite, a sanitary article core and a sanitary item, furthermore chemical products and also the use of this superabsorbent composition in chemical products.

U.S. Pat. Nos. 8,445,596 and 7,407,912 (Mertens) describes crosslinked polymers which are capable of absorbing, which are based on partially neutralized, monoethylenically unsaturated monomers that carry acidic groups, which exhibit improved properties, in particular, with regard to their ability to transport liquids when in a swollen state, and which have been subsequently crosslinked on the surface thereof with a combination consisting of an organic crosslinker compound, with the exception of polyols, and of a cation provided in the form of a salt in an aqueous solution.

Reference to the Figures will assist in a further understanding and appreciation of the invention and its practices. All references cited in the specification are incorporated by reference in their entirety for all disclosure.

FIG. 1 shows a side view of a basic delivery device 2 of the present invention with an indicated degree of expansion 4 upon absorbance of liquid. The device 2 is shown with a carrier layer 6 over a tubular support element 8 having an air passage 10 to allow air to pass through the nostril while the device is implanted in a patient.

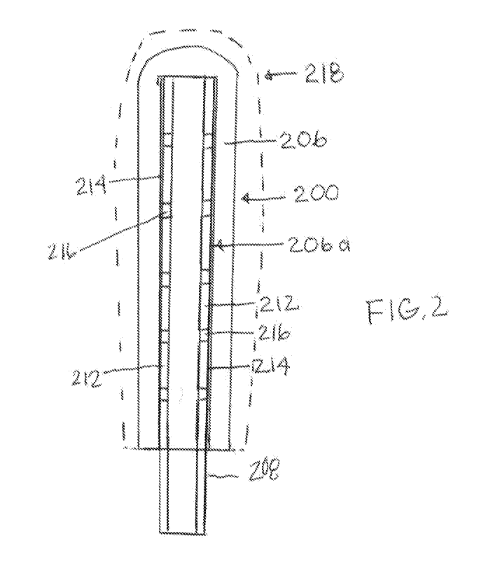

FIG. 2 shows a side view of a delivery device 200 having a fluid delivery conduit 212 before expansion. The device 200 is shown with a carrier layer 206 over a tubular support element 208 and a barrier layer 214 at an inner surface 216 of the carrier layer 206. The fluid delivery conduit 212 delivers the fluid between the barrier layer 214 and the support element 208.

FIG. 3 shows a side view of a delivery device 200 having a fluid delivery 232 conduit after expansion. The delivery device 200 has a fluid delivery conduit 232. The device 200 is shown with a carrier layer 206 over a tubular support element 208 and a barrier layer 214 at an inner surface 216 of the carrier layer 206. The fluid delivery conduit 232 delivers the fluid between the barrier layer 214 and the support element 208. The expanded outer surface 218) of the carrier layer 206 is shown as moved away from the tubular support element 208 creating a pressurizing volume 220 below the barrier layer 214.

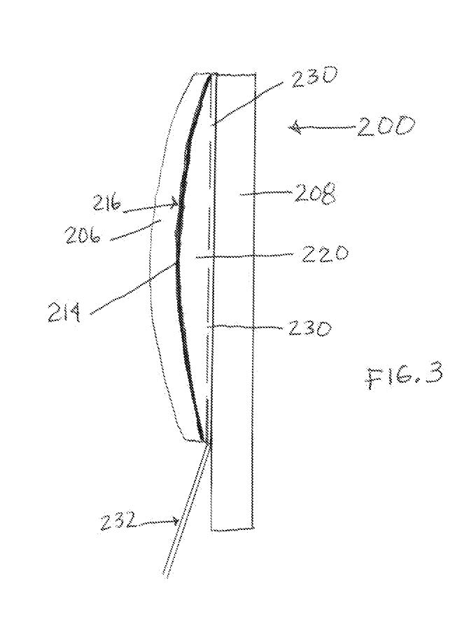

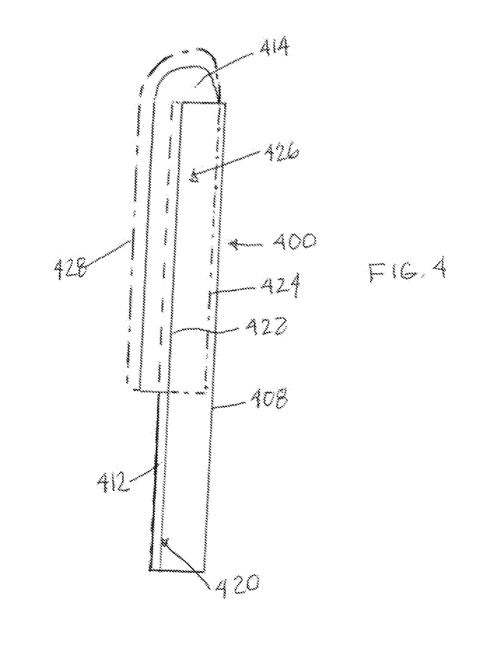

FIG. 4 shows a side view of a delivery device 400 having a fluid delivery conduit 412 before expansion of a partially enclosing carrier layer 414 over a structural support element 408. There is a seal line 420 between the structural support element 408 and the edge end 422 of the carrier layer 414.

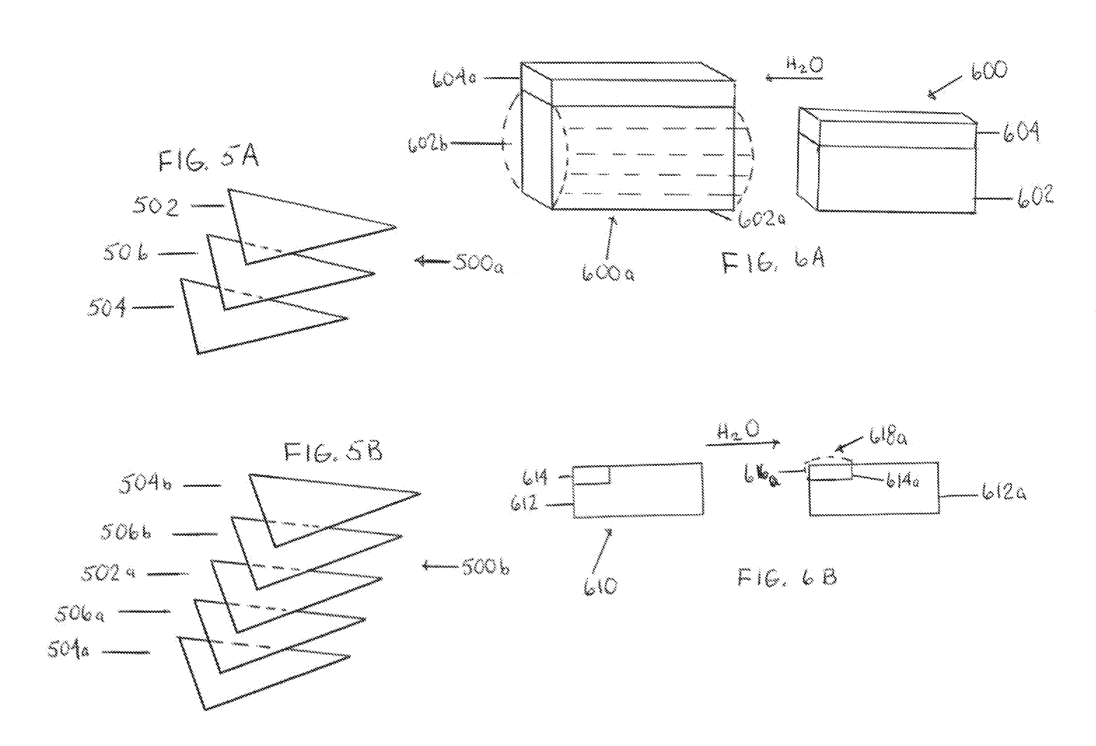

FIG. 5a shows a perspective, exploded view of another insert device 500a structure. This insert device structure 500a is shown with a support layer 502, optional intermediate barrier layer 506, and then a carrier layer 504. An alternative structure depicted in FIG. 5b as structure 500b is shown with a support layer 502a, optional intermediate barrier layers 506a and 506b, and then carrier layers 504a and 504b.

FIG. 6A shows a perspective view of a carrier-support combination 600 before and after addition of water or other liquid. The combination 600 has a support layer 602 and a carrier layer 604. Upon addition of water, the combination 600 swells to form the imbued combination 600a. The imbued combination 600a is shown with the swollen or expanded support layer 602a and the relatively unaffected carrier layer 604a. The expansion shown in the support layer 602a is illustrative for simplicity. In reality, the swelling may be more three-dimensional as illustrated with swollen support layer 602b.

FIG. 6b shows a side view of a controlled dimension carrier-support combination 610 before and after addition of water. The controlled dimension carrier-support combination 610 is illustrated with a support layer 612 and a carrier layer 614 which extends only partly across and into the dimensions of the support layer 612. Upon being imbued with water, the support layer 612 swells to become the imbued support layer 612a with the portion of the carrier layer 614a remaining with the same original dimensions This construction of the carrier-support combination 610 and 610a illustrates how specifically directed pressure and application of dosage within the carrier layer (originally within the carrier layer) is controlled through various embodiments of the present invention.

These and other aspects of the present technology may be varied by design to include other variations and embodiments within the scope of the present technology.

What is claimed:

1. A delivery device for targeted delivery of materials to nasal tissue comprising:
a carrier layer having a first surface and an opposed second surface for providing the material from the first surface of the carrier layer onto the nasal tissue in contact with the first surface of the carrier layer;
a support layer contiguous with at least a portion of the opposed second surface of the carrier layer;
the combination of the carrier layer and the support layer enabling pressure to be exerted or increased between the first surface of the carrier layer and the nasal tissue in contact with the first surface of the carrier layer.

2. The delivery device of claim 1 containing a liquid material the addition of which liquid material delivery to the carrier and/or support layer has expanded volume of the carrier and/or support layer.

3. The delivery device of claim 2 wherein the addition of the liquid material has expanded the volume of the carrier and/or support layer by at least 5% to exert or increase pressure between the first surface of the carrier layer and the nasal tissue.

4. The delivery device of claim 2 wherein the addition of the liquid material has expanded the volume of the carrier and/or support layer by at least 10% to exert or increase pressure between the first surface of the carrier layer and the nasal tissue.

5. The delivery device of claim 1 wherein a conduit for delivery of expanding fluid pressure is provided contiguous to the second surface of the carrier layer.

6. The delivery device of claim 5 wherein the second surface of the carrier layer has a fluid pressure barrier layer adhered thereto and the conduit provides fluid pressure on a side of the fluid pressure barrier layer distal from the second surface of the carrier layer to cause exertion of or increase of pressure between the first surface of the carrier layer and the nasal tissue in contact with the first surface of the carrier layer.

7. The delivery device of claim 6 wherein fluid pressure caused by delivery of a fluid on the distal side of the barrier layer is present between the distal surface of the barrier layer and the support layer.

8. The delivery device of claim 1 wherein the support layer has a first surface and an opposed second surface and at least a portion of the opposed second surface is not in contact with any portion of the carrier layer.

9. The delivery device of claim 1 wherein the support layer has a first surface and an opposed second surface and at least 25% of the opposed second surface is not in contact with any portion of the carrier layer.

10. The delivery device of claim 1 wherein the support layer has a distinct first surface and an opposed distinct second surface having a distinct structural feature separating the distinct first surface and the opposed distinct second surface and at least 25% of the opposed second surface is not in contact with any portion of the carrier layer.

11. The delivery device of claim 10 wherein the distinct structural feature comprises an edge structurally distinguishing between the distinct first surface and the opposed distinct second surface.

12. The delivery device of claim 10 wherein the distinct structural feature comprises a rib structurally distinguishing between the distinct first surface and the opposed distinct second surface.

13. The delivery device of claim 11 wherein the edge divides the support layer into the distinct first surface and the distinct second opposed surface and the distinct first surface comprises between 25% and 80% of total surface area formed by combined total surface areas of the distinct first surface and the opposed distinct second surface.

14. The delivery device of claim 12 wherein the rib divides the support layer into the distinct first surface and the distinct second opposed surface and the distinct first surface comprises between 25% and 80% of total surface area formed by combined total surface areas of the distinct first surface and the opposed distinct second surface.

15. The delivery device of claim 6 wherein the support layer has a distinct first surface and an opposed distinct second surface having a distinct structural feature separating the distinct first surface and the opposed distinct second surface and at least 25% of the opposed second surface is not in contact with any portion of the carrier layer.

16. The delivery device of claim 15 wherein the distinct structural feature comprises an edge structurally distinguishing between the distinct first surface and the opposed distinct second surface.

17. The delivery device of claim 15 wherein the distinct structural feature comprises a rib structurally distinguishing between the distinct first surface and the opposed distinct second surface.

18. The delivery device of claim 16 wherein the edge divides the support layer into the distinct first surface and the distinct second opposed surface and the distinct first surface comprises between 25% and 80% of total surface area formed by combined total surface areas of the distinct first surface and the opposed distinct second surface.

19. The delivery device of claim 17 wherein the rib divides the support layer into the distinct first surface and the distinct second opposed surface and the distinct first surface comprises between 25% and 80% of total surface area formed by combined total surface areas of the distinct first surface and the opposed distinct second surface.

20. The delivery device of claim 15 wherein the structural feature separates a zone contiguous with the first distinct surface of the support layer within which the fluid pressure expanding fluid pressure is provided to expand the carrier layer or cause the carrier layer to exert pressure against the nasal tissue.

21. The delivery device of claim 16 wherein the structural feature separates a zone contiguous with the first distinct surface of the support layer within which the fluid pressure expanding fluid pressure is provided to expand the carrier layer or cause the carrier layer to exert pressure against the nasal tissue.

22. The delivery device of claim 17 wherein the structural feature separates a zone contiguous with the first distinct surface of the support layer within which the fluid pressure expanding fluid pressure is provided to expand the carrier layer or cause the carrier layer to exert pressure against the nasal tissue.

23. The delivery device of claim 18 wherein the structural feature separates a zone contiguous with the first distinct surface of the support layer within which the fluid pressure expanding fluid pressure is provided to expand the carrier layer or cause the carrier layer to exert pressure against the nasal tissue.

24. The delivery device of claim 19 wherein the structural feature separates a zone contiguous with the first distinct surface of the support layer within which the fluid pressure expanding fluid pressure is provided to expand the carrier layer or cause the carrier layer to exert pressure against the nasal tissue.

25. A method of delivery material to be absorbed into or through nasal tissue comprising adding material to a delivery device comprising:
a carrier layer having a first surface and an opposed second surface for providing the material from the first surface of the carrier layer onto the nasal tissue in contact with the first surface of the carrier layer;
a support layer contiguous with at least a portion of the opposed second surface;
the combination of the carrier layer and the support layer enabling pressure to be exerted or increased between the first surface of the carrier layer and the nasal tissue in contact with the first surface of the carrier layer;
inserting the delivery device into a nasal passage and exerting pressure or increasing pressure between the first surface of the carrier layer and the nasal tissue while material to be delivered is present within the carrier layer.

26. The method of claim 25 wherein the pressure is exerted or increased by addition of fluid into the carrier and/or support layer.

27. The method of claim 26 wherein the fluid added to the carrier layer comprises material carried in the fluid to be delivered to the nasal tissue.

28. The method of claim 26 wherein the fluid added to the carrier layer assists delivery of material already within the carrier layer before addition of the liquid onto or into the nasal tissue.

29. The method of claim 27 wherein the material comprises medically active ingredients.

30. The method of claim 28 wherein the material comprises medically active ingredients.

31. The method of claim 27 wherein the medically active ingredients is selected from the group consisting of analgesics, antibiotics, antifungal agents, antihistamines, NSAIDS, steroids, botulinum toxin, Clostridia sp derived neurotoxin, vaccine, viruses, bacteria, liposomes, noisomes and tetanus toxins.

32. The method of claim 28 wherein the medically active ingredients is selected from the group consisting of analgesics, antibiotics, antifungal agents, antihistamines, NSAIDS, steroids, botulinum toxin, Clostridia sp derived neurotoxin, vaccine, viruses, bacteria, liposomes, noisomes and tetanus toxins.

33. The delivery device of claim 1 comprising an expanding element that provides force against an interior surface of the carrier layer to expand volume of the carrier layer or to provide contact force between the carrier layer and the nasal tissue.

34. The delivery device of claim 33 wherein the expanding element is selected from the group consisting of inflatable elements, elements with an expansive elastic memory, and clip-like elements where applied force to one segment of the clip-like element expands another segment of the element.
